(19) United States
(12) Patent Application Publication
(10) Pub. No.: US 2008/0269765 A1
(43) Pub. Date: Oct. 30, 2008

(54) INSTRUMENT AND METHOD FOR IMPLANTING A PROSTHETIC COMPONENT

(75) Inventors: Barish Banerjee, Kolkata (IN); Stephen J. Vankoski, Fort Wayne, IN (US)

Correspondence Address: BAKER & DANIELS LLP;111 E. WAYNE STREET, SUITE 800, FORT WAYNE, IN, 46802, US

(73) Assignee: ZIMMER, INC., Warsaw, IN (US)

(21) Appl. No.: 11/740,988

(22) Filed: Apr. 27, 2007

(51) Int. Cl. A61B 17/58

(52) U.S. Cl. 606/99

(57) ABSTRACT

A surgical instrument including an inserter having a body and a head. The head is dimensioned for receipt within a cavity formed by a final junction surface, for example a female tapered surface, of a prosthetic component. In one exemplary embodiment, a portion of the head has a scratch hardness that is less than the scratch hardness of the prosthetic component, i.e., is formed from a softer material. This allows for the cover of the head to be received within the cavity formed by the final junction surface of the prosthetic component without substantially scratching or damaging the final junction surface.

### BACKGROUND

1. Field of the Invention

The present invention relates to a surgical instrument, and, particularly, to a surgical instrument for facilitating the implantation of a prosthetic component.

2. Description of the Related Art

Prostheses are commonly utilized to repair and/or replace damaged bone and tissue in the human body. For example, a hip prosthesis may be implanted to replace damaged or destroyed bone in the femur and/or acetabulum and recreate the natural, anatomical articulation of the hip joint. To implant a prosthesis, orthopedic surgery is performed, which, in some cases, may be a minimally invasive surgery.

To facilitate a minimally invasive surgery, a modular prosthesis may be used. Modular prostheses have several individual, distinct components that are connected together to form a final, implanted prosthesis. To connect the components, such as a femoral stem and a femoral neck in a modular femoral prosthesis system, each of the components may include a final junction surface. The final junction surfaces may form corresponding Morse tapers, for example. During the implantation of the individual components of the modular prosthesis, several different instruments and/or additional components, such as an inserter instrument or a provisional prosthetic component, may come in contact with the final junction surfaces of the modular prosthetic components.

### SUMMARY

The present invention relates to a surgical instrument, and, particularly, to a surgical instrument for facilitating the implantation of a prosthetic component. In one exemplary embodiment, the surgical instrument includes an inserter having a body and a head. The head is dimensioned for receipt within a cavity formed by a final junction surface, for example a female tapered surface, of the prosthetic component. In one exemplary embodiment, a portion of the head has a scratch hardness that is less than the scratch hardness of the prosthetic component, i.e., is formed from a softer material. This allows for the head to be received within the cavity formed by the final junction surface of the prosthetic component without risking damage to the final junction surface during impaction of the prosthetic component.

In another exemplary embodiment, the surgical instrument of the present invention further includes a force transfer component that may be actuated to extend from the body of the inserter. In this embodiment, with the head of the inserter received within a cavity formed by the final junction surface of the prosthetic component, the force transfer component may be actuated to contact the end of the prosthetic component at a location apart from the final junction surface. When the force transfer component is in contact with the prosthetic component, an impaction force applied to the surgical instrument is transferred via the force transfer component to the prosthetic component.

Advantageously, by utilizing the force transfer component to transfer an impaction force to the prosthetic component at a location apart from the final junction surface of the prosthetic component, the direct application of the impaction force to the final junction surface is substantially avoided. As a result, any scratching or additional damage to the final junction surface that may occur as a result of the receipt of the impaction force is substantially eliminated.

In one form thereof, the present invention provides an instrument for facilitating the insertion of a prosthetic component, the prosthetic component having a longitudinal axis and a cavity defined by a final junction surface into the body of a patient, the instrument including: an inserter having an elongate body and a head; the head having a core and a cover overlying the core, the cover having an insertion junction surface dimensioned for receipt within the cavity defined by the final junction surface of the prosthetic component, the cover formed from a cover material, the final junction surface formed from a final junction material, the cover material having a lower scratch hardness than the final junction material; whereby interaction of the cover material with the final junction material will more likely cause scratching of the cover material than the final junction material; an impaction surface; and a force transfer component connected to the impaction surface, the force transfer component positionable adjacent the prosthetic component, whereby force applied to the impaction surface is transferred to the prosthetic component via the force transfer component.

In another form thereof, the present invention provides a combination including: a prosthetic component having a longitudinal axis and a cavity defined by a final junction surface, the final junction surface formed from a final junction surface material; and an inserter having an elongate body and a head; the head having a core and a cover overlying the core, the cover having an insertion junction surface dimensioned for receipt within the cavity defined by the final junction surface of the prosthetic component, the cover formed from a cover material, the cover material having a lower scratch hardness than the final junction material; whereby interaction of the cover material with the final junction material will more likely cause scratching of the cover material than the final junction material; an impaction surface; a force transfer component connected to the impaction surface, the force transfer component positionable adjacent the prosthetic component, whereby force applied to the impaction surface is transferred to the prosthetic component via the force transfer component.

In yet another form thereof, the present invention provides a method of inserting a prosthetic component having a longitudinal axis into the body of a patient, including the steps of: attaching an inserter to a prosthetic component, the inserter comprising: a head and an elongate body, the elongate body defining a longitudinal axis, the head having a core and a cover overlying the core, the cover having an insertion junction surface dimensioned to mate with a final junction surface of the prosthetic component, the cover formed from a cover material, the final junction surface formed from a final junction material, the cover material having a lower scratch hardness than the final junction material; placing a force transfer component having a longitudinal axis in a position to transfer force to the prosthetic component; and impacting the inserter, whereby a force is transferred from the force transfer component to the prosthetic component, to seat the prosthetic component within the body of the patient.

### BRIEF DESCRIPTION OF THE DRAWINGS

The above-mentioned and other features and advantages of this invention, and the manner of attaining them, will become more apparent and the invention itself will be better understood by reference to the following description of an embodiment of the invention taken in conjunction with the accompanying drawings, wherein:

FIG. 1 is a perspective view of an instrument including an impaction device and inserter in accordance with one form of the present invention;

FIG. 2 is a partial cross-sectional view of the instrument of FIG. 1 depicting the inserter secured to a prosthetic component with the force transfer device in the non-engagement position;

FIG. 3 is a partial, cross-sectional view of the instrument of FIG. 1 depicting the inserter secured to a prosthetic component with the force transfer device in the engagement position; and

FIG. 4 is an enlarged fragmentary view of FIG. 2.

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION

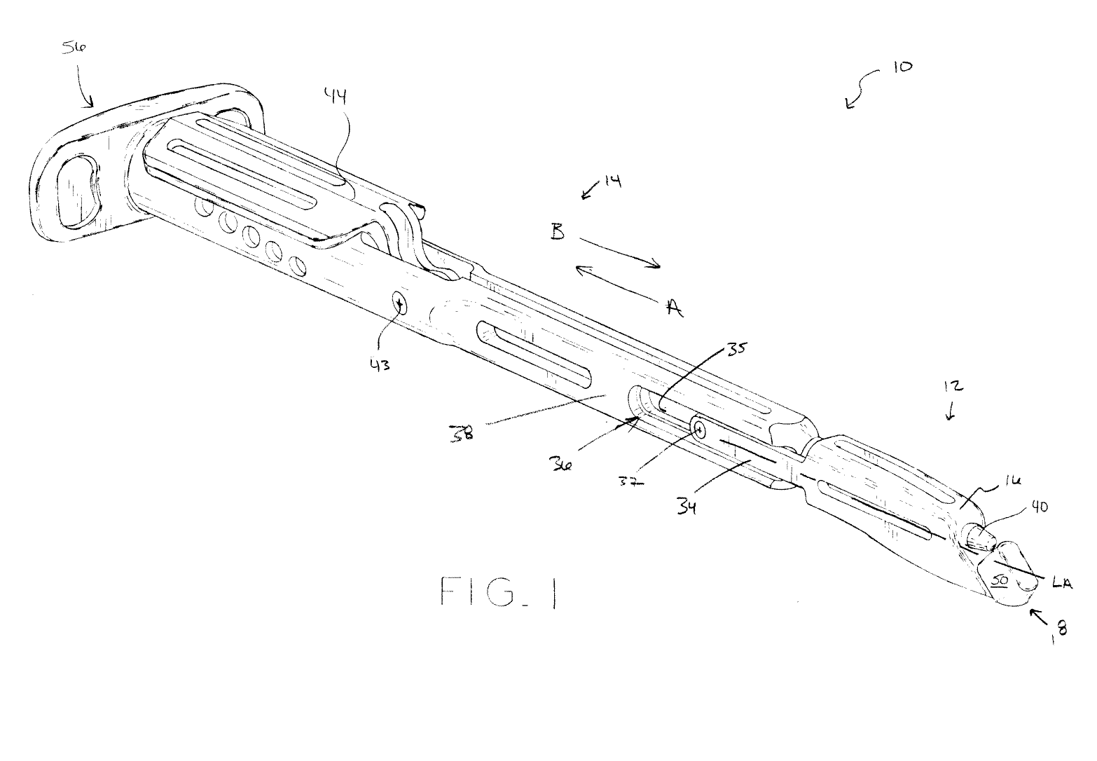

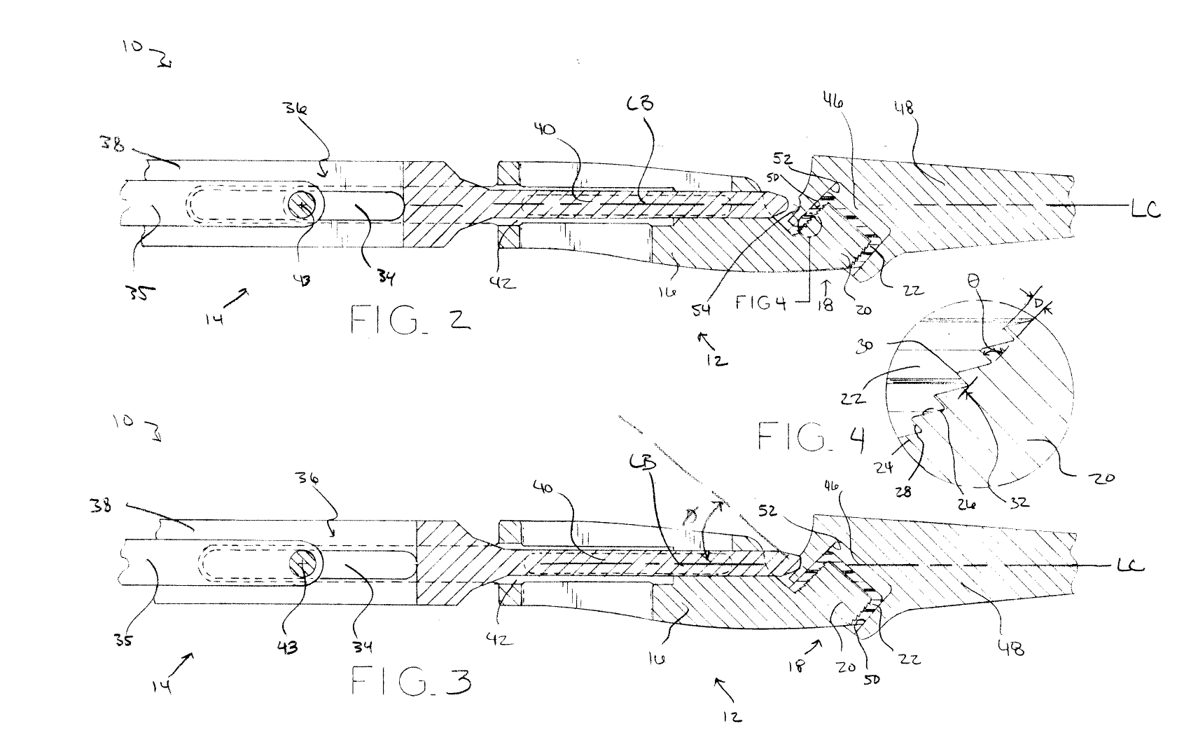

Referring to FIGS. 1-3, instrument 10 includes inserter 12 and impaction device 14. Inserter 12 includes body 16, defining longitudinal axis LA, and head 18. Head 18 has an internal core 20 and cover 22 overlying core 20 (FIGS. 2 and 3). Cover 22 is formed of a material having a lower scratch hardness than femoral stem 48. Instrument 10 further includes force transfer component 40, which is described in detail below.

Cover 22 may be secured to core 20 in any known manner. In one exemplary embodiment, cover 22 is thermally assembled to core 20. Specifically, cover 22 and core 20 are heated and pressure is applied to cover 22 to interdigitate cover 22 with teeth 24, described below, of core 20. In other exemplary embodiments, cover 22 may be overmolded directly onto core 20 or, alternatively, cover 22 may be formed as a disposable cap designed to friction fit over core 20. Additionally, epoxy may be used, either alone or in combination with any of the above methods, to secure cover 22 to core 20.

In one exemplary embodiment, core 20 includes a plurality of teeth 24, shown in FIG. 4, extending from the exterior thereof. Referring to FIG. 4, teeth 24 are formed by distal flanks 26 and proximal flanks 28. Teeth 24 have a depth D extending from crest 30 to root plane 32. In one exemplary embodiment, teeth 24 have a depth D substantially equal to or greater than nine thousandths of an inch (0.009″), which helps to retain cover 22 on core 20 during impaction and/or extraction of a prosthetic component, as described in detail below. In one exemplary embodiment, teeth 24 have a proximal flank angle θ greater than 45°, which provides additional retention of cover 22 on core 20. Specifically, referring to FIG. 4, the proximal flank angle θ is the angle between proximal flanks 28 and root planes 32 measured in the direction of the distal flanks 26.

As shown in FIGS. 1-3, inserter 12 further includes opposing arms 34 extending from body 16. Arms 34 are configured to be received within slot 36 formed in body 38 of impaction device 14. Specifically, linkage 35 of inserter 12 is received between opposing arms 34 of impaction device 14. Arms 34 are then rotatably secured to linkage 35 in any known manner, such as by removable pin 37. With arms 34 of inserter 12 positioned within slot 36 of impaction device 14 and secured to linkage 35 thereof, force transfer component 40, depicted herein as a rod extending from impaction device 14, extends through aperture 42 in body 16 of inserter 12. While described and depicted herein as secured to and extending from impaction device 14, force transfer component 40 may be secured to inserter 12 and impaction device 14 may be entirely absent.

With inserter 12 connected to impaction device 14 as described above, actuation of actuation mechanism 44 of impaction device 14 about hinge point 43 results in movement of inserter 12 longitudinally in slot 36. Specifically, actuation of actuation mechanism 44 converts the rotational movement of actuation mechanism 44 about hinge point 43 into translational movement of linkage 35 along slot 36. Thus, actuation mechanism 44 may be connected to linkage 35 in any known manner which facilitates this conversion. For example, actuation mechanism 44 may be connected to linkage 35 via a cam mechanism.

As shown in FIGS. 1 and 3, actuation mechanism 44 is in the actuated position, resulting in displacement of inserter 12 along slot 36 in the direction of arrow A. In contrast, as shown in FIG. 2, actuation mechanism 44 is in the unactuated position, resulting in displacement of inserter 12 along slot 36 in the direction of arrow B of FIG. 2. Thus, actuation of actuation mechanism 44 and, correspondingly, inserter 12, moves force transfer component 40 between a first, non-engagement position (FIG. 2) and a second, engagement position (FIGS. 1 and 3), as described in detail below.

In another exemplary embodiment, inserter 12 is fixedly secured to impaction device 14 and actuation of actuation mechanism 44 results in movement of force transfer component 40. Thus, inserter 12 remains in a fixed position relative to impaction device 14 and movement of force transfer component 40 places the same in the engagement and non-engagement positions described above. In this embodiment, force transfer component 40 may be rotatably secured to linkage 35 to effect movement thereof.

Instrument 10, as set forth above, is utilized to facilitate the insertion and/or extraction of a prosthetic component. Specifically, referring to FIGS. 2 and 3, head 18 of inserter 12 is configured to be received within the junction of a prosthetic component, such as final junction 46 of femoral stem 48. Femoral stem 48 is one component of a modular femoral prostheses and final junction 46 thereof is dimensioned to taper lock with a male tapered portion extending from a femoral head and/or femoral neck component (not shown). As shown, insertion junction surface 50 of cover 22 of head 18 is dimensioned for receipt within the cavity formed by final junction surface 52 of junction 46 of femoral stem component 48. Thus, the interaction between insertion junction surface 50 and final junction surface 52 provides a connection between inserter 12 and femoral stem 48. Additionally, during implantation of the modular femoral prosthesis, other instruments and components, such as the provisional component of U.S. patent application Ser. No. 11/538,828, filed Oct. 5, 2006, entitled PROVISIONAL PROSTHETIC COMPONENT FORMED OF MULTIPLE MATERIALS, the entire disclosure of which is expressly incorporated by reference herein, may come in contact with final junction 46.

To help protect final junction 46 during the implantation of femoral stem component 48, head 18 includes cover 22 and core 20. Cover 22 may be constructed from a material having a scratch hardness that is less than the scratch hardness of femoral stem 48. Scratch hardness refers to the resistance of a material to penetration, i.e., scratching, by other materials. Thus, a material having a high scratch hardness can penetrate, i.e., scratch, a material having a lower scratch hardness. Similarly, a material having a lower scratch hardness is less likely to penetrate, i.e., scratch, a material having a higher scratch hardness. By having a lower scratch hardness, cover 22 is prevented from scratching or damaging the walls defining final junction 46 of femoral stem 48 when inserted therein. For example, when femoral stem 48 is constructed of titanium or a titanium alloy, cover 22 may be constructed of a suitable plastic having a lower scratch hardness than titanium or titanium alloy, such as carbon fiber polyetheretherketone, polyethelyene, polytetrafluoroethylene, polyphenylsulfone, such as Radel® A, or polyethersulfone, such as Radel® B. Radel® is a registered trademark of Amoco Polymers, Inc. of Alpharetta, Ga. In one exemplary embodiment, cover 22 has a scratch hardness substantially less then the scratch hardness of core 20, for example, when cover 22 is made of polyethersulfone, such as Radel® A having a Rockwell hardness of 122, and core 20 is made of titanium, having a Rockwell hardness of 30-35.

With inserter 12 and, correspondingly, impaction device 14, connected to femoral stem component 48 via head 18 of inserter 12, movement of inserter 12 results in corresponding movement of the femoral stem 48. To facilitate the connection of instrument 10 to femoral stem 48 and the impaction of femoral stem 48 at the implantation site, actuator mechanism 44 is moved from the unactuated position to the actuated position (FIG. 1) and, correspondingly, body 16 of inserter 12 is moved in the direction of arrow A, as described in detail above. As a result of the movement of body 16, femoral stem 48, which is connected to head 18 at junction 46, is correspondingly moved. Specifically, femoral stem component 48 is advanced until force transfer component 40 contacts dimple 54 of femoral stem component 48. Instrument 10 and femoral stem 48 may then be aligned with and advanced toward the implantation site.

With force transfer component 40 in contact with dimple 54 of femoral stem 48, force transfer component 40 is in the engagement position. In this position, impaction force applied to impaction surface 56 of impaction device 14 is transferred via force transfer component 40 to femoral stem component 48 via dimple 54. In certain exemplary embodiments, longitudinal axis LB of force transfer component 40 and longitudinal axis LC of femoral stem 48 are collinear or parallel. In certain other exemplary embodiments, longitudinal axes LB, LC form an angle θ (FIG. 3) that is less than forty-five degrees (45°).

Advantageously, by transferring the impaction force to dimple 54 of femoral stem component 48, substantially none of the impaction force is transferred directly to junction 46. Thus, the potential for head 18 scratching or otherwise damaging final junction surface 52 is substantially lessened. Moreover, femoral stem 48 may have greater structural strength near the location of dimple 54 and/or may transfer the impaction force throughout femoral stem 48 in a desirable manner. Once femoral stem component 48 is properly positioned within a patient's body, actuation mechanism 44 of impaction device 14 may be moved to the unactuated position and head 18 of inserter 12 unseated from junction 46 of femoral stem component 48. Instrument 10 may then be removed from the patient's body.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An instrument for facilitating the insertion of a prosthetic component, the prosthetic component having a longitudinal axis and a cavity defined by a final junction surface into the body of a patient, the instrument comprising:
an inserter having an elongate body and a head;
said head having a core and a cover overlying said core, said cover having an insertion junction surface dimensioned for receipt within the cavity defined by the final junction surface of the prosthetic component, said cover formed from a cover material, the final junction surface formed from a final junction material, said cover material having a lower scratch hardness than the final junction material;
whereby interaction of said cover material with the final junction material will more likely cause scratching of said cover material than the final junction material;
an impaction surface; and
a force transfer component connected to said impaction surface, said force transfer component positionable adjacent the prosthetic component, whereby force applied to said impaction surface is transferred to the prosthetic component via said force transfer component.

2. The instrument of claim 1, wherein said cover material has a substantially lower scratch hardness than the final junction material.

3. The instrument of claim 1, wherein said cover material is a plastic.

4. The instrument of claim 3, wherein said plastic is selected from the group consisting of: polyetheretherketone, carbon fiber polyetheretherketone, polyethelyene, polytetrafluoroethylene, polyphenylsulfone, and polyethersulfone.

5. The instrument of claim 3, wherein said core of said head is a metal.

6. The instrument of claim 1, wherein said core further includes an exterior surface having a plurality of teeth formed thereon, said teeth configured to facilitate securement of said cover to said core.

7. The instrument of claim 6, wherein said teeth have a depth greater than nine thousandths of an inch.

8. The instrument of claim 6, wherein each of said teeth has a proximal flank and a distal flank, each said proximal flank of said plurality of teeth having a proximal flank angle greater than forty-five degrees.

9. The instrument of claim 1, wherein said inserter defines a longitudinal axis, said head angularly offset relative to said longitudinal axis.

10. The instrument of claim 1, further comprising an actuation mechanism moveable between an unactuated position and an actuated position, wherein movement of said actuation mechanism between said unactuated position and said actuated position results in corresponding movement of at least one of said inserter and said force transfer component from a first, non-engagement position to a second, engagement position in which said force transfer component is capable of transferring force to the prosthetic component.

11. A combination comprising:
a prosthetic component having a longitudinal axis and a cavity defined by a final junction surface, said final junction surface formed from a final junction surface material; and
an inserter having an elongate body and a head;
said head having a core and a cover overlying said core, said cover having an insertion junction surface dimensioned for receipt within said cavity defined by said final junction surface of said prosthetic component, said cover formed from a cover material, said cover material having a lower scratch hardness than said final junction material;
whereby interaction of said cover material with said final junction material will more likely cause scratching of said cover material than said final junction material;
an impaction surface;
a force transfer component connected to said impaction surface, said force transfer component positionable adjacent said prosthetic component, whereby force applied to said impaction surface is transferred to said prosthetic component via said force transfer component.

12. The combination of claim 11, further comprising an actuation mechanism moveable between an unactuated position and an actuated position, wherein movement of said actuation mechanism between said unactuated position and said actuated position results in corresponding movement of at least one of said inserter and said force transfer component from a first, non-engagement position to a second, engagement position in which said force transfer component is capable of transferring force to said prosthetic component.

13. The combination of claim 11, wherein said cover material has a substantially lower scratch hardness than said final junction material.

14. The combination of claim 11, wherein said cover material is a plastic.

15. The combination of claim 14, wherein said plastic is selected from the group consisting of: polyetheretherketone, carbon fiber polyetheretherketone, polyethelyene, polytetrafluoroethylene, polyphenylsulfone, and polyethersulfone.

16. The combination of claim 11, wherein said core of said head further includes an exterior surface having a plurality of teeth formed thereon, said teeth configured to facilitate securement of said cover to said core.

17. A method of inserting a prosthetic component having a longitudinal axis into the body of a patient, comprising the steps of:
attaching an inserter to a prosthetic component, the inserter comprising:
a head and an elongate body, said elongate body defining a longitudinal axis, said head having a core and a cover overlying said core, said cover having an insertion junction surface dimensioned to mate with a final junction surface of the prosthetic component, said cover formed from a cover material, the final junction surface formed from a final junction material, said cover material having a lower scratch hardness than the final junction material;
placing a force transfer component having a longitudinal axis in a position to transfer force to the prosthetic component; and
impacting the inserter, whereby a force is transferred from the force transfer component to the prosthetic component, to seat the prosthetic component within the body of the patient.

18. The method of claim 17, further comprising the step of placing the force transfer component out of position to transfer force to the prosthetic component.

19. The method of claim 17, wherein the placing step further includes moving an actuation mechanism to place the force transfer component in a position to transfer force to the prosthetic component.
